(19) United States
(12) Patent Application Publication
(10) Pub. No.: US 2015/0177206 A1
(43) Pub. Date: Jun. 25, 2015

(54) GAS SENSOR COMPRISING FLAMEPROOF BARRIER

(71) Applicant: Crowcon Detection Instruments Limited, Abingdon, Oxfordshire (GB)

(72) Inventors: Paul Basham, Abingdon (GB); Roger Hutton, Abingdon (GB)

(21) Appl. No.: 14/409,749

(22) PCT Filed: Jan. 21, 2013
(86) PCT No.: PCT/GB2013/050129

§ 371 Date: Dec. 19, 2014

(30) Foreign Application Priority Data
Jun. 19, 2012 (GB) 1210846.0

(51) Int. Cl. G01N 33/00

(52) U.S. Cl. CPC G01N 33/0009

(57) ABSTRACT

A flameproof barrier for use in a gas sensor or gas detector, the flameproof barrier comprising: a porous element through which gas passes, wherein the porous element is coated with a layer, the material of the layer chosen so as to promote gas transport through the porous element of the flameproof barrier and to limit corrosion and to remain unblocked from environmental contaminants.

### CROSS-REFERENCE TO RELATED APPLICATION

This application is a United States national phase application based on PCT/GB2013/050129 filed on Jan.21, 2013, which claims the benefit of British Patent Application No. GB 1210846.0 filed on Jun. 19, 2012, the entire disclosures of which are hereby incorporated herein by reference.

### TECHNICAL FIELD

The present invention relates to improvements in flameproof barriers, in particular those used in gas sensors or detectors.

### BACKGROUND TO THE INVENTION

Gas Detectors are typically placed in hazardous environments. Typically such environments may contain explosive or flammable gases. Therefore, it is important that in such gas detectors the components are intrinsically safe so as to avoid the ignition of gases or that the ability of a fire, or explosion, to propagate is minimised.

It is known in commercially available products to protect such components by way of intrinsically safe electronics. Such electronics are typically costly and therefore contribute to increased production costs.

An alternative method known in commercially available products is to provide a flameproof barrier known as a sinter or flame arrestor. Such flameproof barriers are placed in the likely path of a flame, and are typically in the form of a porous, or mesh, structure, often made of a metallic or ceramic material. When the flame passes to the flameproof bather, the porous nature of the barrier prevents the flame propagating as heat from the flame is conducted away by the sinter causing the flame eventually to be extinguished.

Whilst such sinters are effective as acting as a barrier to the transmission of flames, by their very nature they also result in a reduction of the amount of gas which can pass through the sinters. It is found that the transport of gases to be detected, such as hydrogen sulphide, hydrogen chlorine, ozone, phosphine etc., are attenuated as they pass through the sinters thereby reducing the sensitivity of the gas sensor as well as increasing the response time of the sensor.

Furthermore, such sinters may also prevent the transport of gases which are of interest to a user therefore preventing the detection of such gases.

In order to mitigate at least some of the above mentioned problems, there is provided a flameproof barrier for use in a gas sensor or gas detector, the flameproof barrier comprising: a porous element through which gas passes, wherein the porous element is coated with a protective layer, such as a hydrophobic layer, so as to promote gas transport through the porous element of the flameproof barrier.

Such a coating promotes gas transport through the porous sinter or flame arrestor element improving the response time of the sensor, as well as allowing gases which are not normally detectable in a gas detector with a sinter to be detected.

Preferably, the layer is less than 5pm in thickness, preferably less than 1 μm, more preferably of the order of nanometre thickness. Preferably, wherein the layer is applied to the porous element as a plasma coating, or wherein the layer is applied to the porous element using electrolytic deposition metal organic chemical deposition (MOCVD) or chemical vapour deposition (CVD) methods.

Preferably, wherein the layer is selected from a group comprising SiO, 1H, 1H, 2H, 2H-heptadecafluorodecylacrylate, titanium dioxide, silicon monoxide, tin dioxide and indium tin oxide or their chemical analogue. Preferably, wherein the protective layer is selected such that it promotes the transport of one or more of the following gases: H₂S, SO₂, NO, PH₃, O₃, HCl, HCN and THF. Preferably, wherein the porous element is a porous sinter or flame arrestor.

The invention also describes a gas detector or gas sensor comprising the flameproof barrier described herein. Further aspects of the invention will be apparent from the appended claim set.

### BRIEF DESCRIPTION OF THE FIGURES

Embodiments of the invention are now described, by way of example only, with reference to the accompanying drawing in which:

FIG. 1 is a schematic representation of the invention.

### DETAILED DESCRIPTION OF AN EMBODIMENT

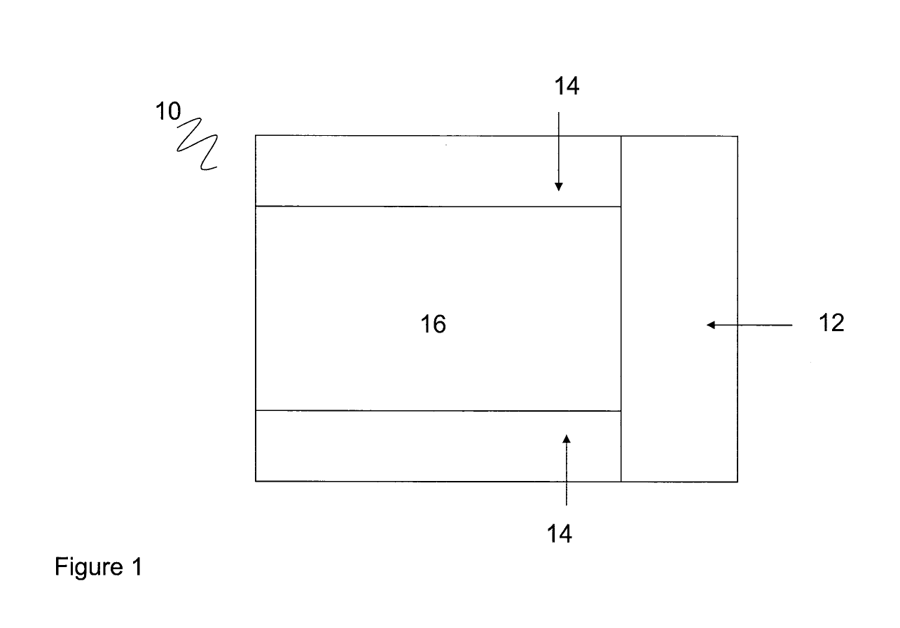

FIG. 1 is a schematic representation of an example of the invention. There is shown a gas detector 10, the gas detector 10 comprising: a gas sensor 12 configured to detect the presence of one or more target gases within an atmosphere, the sensor 12 further comprising processing means to determine the amount of gas present (not shown); a sinter housing 14; the sinter housing comprising a flameproof bather such as a flame arrestor or sinter 16. In the present specification the terms flame arrestor and sinter are used interchangeably and refer to any form of porous flameproof barrier.

In use, the gas detector 10 is placed in the environment to be tested, and gas from the atmosphere to be tested passes through the sinter 16 which is contained in the sinter housing 14 and continues to the sensor 12. The sinter 16, in an example, is a porous metallic or ceramic structure which is approximately 5 millimetres thick and is made of a material with pore sizes of approximately 10 to 100 microns in diameter.

The gas in the atmosphere usually comprises a mixture of “normal” atmospheric gas, explosive gases such as methane, and toxic gases such as carbon monoxide, hydrogen sulphide, ozone and ETO (Ethylene oxide) must all pass through the sinter in order to reach the gas sensor 12. Preferably, the sinter housing 14 in which the sinter 16 is held is positioned over the sensor 12, and accordingly any gas which is detected by the sensor 12 must pass through the sinter 16. The sensor 12 then detects the gas in the known manner.

As the sinter 16 is made of a porous material, the transport of gas through the sensor sinter 16 may result in an increased response time and or sluggish detector response or intermittent response.

In order to mitigate the problems associated with the reduced sensitivity and the increased response times and intermittent response due to the presence of the sinter 16, there is provided an improved flameproof barrier or sinter.

According to an example of the invention, in order to improve the gas transport through the gas detector 10, and in particular the sinter 16, (which in a typical gas sensor is approximately 5 mm thick) the sinter 16 is coated with a protective layer, such as a hydrophobic material, in order to enhance gas transport through the sinter 16. It has been beneficially realised that it is possible to ensure that the flameproof barrier/ sinter 16 may still act as an effective flameproof barrier whilst simultaneously promoting gas transport through the sinter 16 (thereby ensuring that the sensor 12 can detect the presence of one or more target gases in an efficient manner) by coating the sinter in a hydrophobic or protective material. It has been found that by coating the sinter with a material gas transport properties of the sinter are enhanced, reducing the time taken for a gas to traverse the sinter 16. Preferably, the coating applied to the sinter is of the order of nanometre thickness, though in further examples of the invention the coating may be microns in thickness. Such a thickness is preferred as it ensures that the pores of the sinter do not become blocked, thereby negating any benefit associated with coating.

In an example, the coating is applied to the sinter 16 using a plasma coating method. Plasma coating methods are known in the art and are commercially available. The use of plasma coating is particularly beneficial as it provides a cost effective mechanism for coating the sinter 16 with a coating of the order of a nanometre thickness.

In further embodiments the coating may be applied to the sinter using an electrolytic deposition metal organic chemical deposition method (MOCVD) or a chemical vapour deposition method (CVD). In further examples, other known methods for applying coatings on a substrate of the order of nanometres to microns may be used.

The plasma coating method, or other deposition methods, deposit the layer of the material on the inner and outer surfaces of the sinter 16, thereby improving the gas transport property of the sinter. The coating of the sinter 16 with the layer results in a demonstrable improvement in gas transport for gases including H₂S, SO₂, NO, PH₃, O₃, HCl, HCN and THF. Some of these gases are not normally detectable in known gas detectors which include a sinter as the flameproof barrier, as the barrier prevents the efficient transport of such gases through the sinter, causing the gas to be undetected by the sensor 12.

For example, H₂S is not detected in many commercially available gas detectors which include a sinter 16. When the sinter 16 is coated with the protective layer, such as hydrophobic layer, it is found that H2S may be detected by the same detector. Similarly, SO₂ is not detected in many commercially available gas detectors which include a sinter 16. When the sinter 16 is coated with the protective layer, it is found that SO₂ may be detected by the same detector. In this example, the protective layer is particularly advantageous as the coating forms a protective layer against the SO₂, limiting corrosion of the sinter material.

Therefore the present invention allows for a larger variety of gases, than would normally be expected, to be detected in some commercially available gas detectors. Furthermore, the improved gas transport results in a shorter crossing time for the gas to cross across the sinter 16 thereby increasing the improving throughput as well as the reducing the response time of the detector 10 and sensor 12.

It is also found that a further advantage of the application of the layer to the detector 10 is that it provides a further layer of protection against many hazardous materials which are typically found in the environments in which gas detectors 10 are placed. The protective layer provides protection against various environmental and chemical elements such as salts, greases, foreign bodies etc. which are typically damaging to a gas detector. Such environmental and chemical elements are typically found in humid and polluted and marine environments in which a gas detector is may be placed. Therefore, as well as improving the throughput of the sensor, reducing response time, and increasing sensitivity, the coating of the sinter 16 also increases the lifetime of the sensor by reducing the effects of corrosion etc., on the coated elements.

In a preferred example, further elements of the gas detector are also coated with a layer. For example, the surfaces which are typically found to be in contact with the gases, e.g. pipes, pumps, supports, etc., are also coated with the layer. This allows the improved gas transport properties to occur all through the gas detector, and furthermore reduces the effect of the environmental and chemical elements which are known to be detrimental to the gas detectors.

Therefore, the present invention increases the lifetime of the detector and reduces maintenance costs.

In a preferred embodiment, in an example, the sinter 16 (and indeed other elements of the gas detector) is plasma coated with a protective layer of one of the group selected from: SiO, 1H, 1H, 2H, 2H-heptadecafluorodecylacrylate, titanium dioxide, silicon monoxide, tin dioxide and indium tin oxide or their chemical analogues. Such materials are preferred, as they have been found by the applicant to improve gas transports through the sinter layer. In further examples, other suitable coatings may be used.

Accordingly, the present invention therefore provides an improved flameproof bather or flame arrestor, in which the barrier is coated with a layer of material in order to promote gas transport through the flameproof barrier. This results in the increase in throughput of the gas detector, and for the gas detector to detect one or more gases which under normal circumstances may not be detected due to the attenuation of the gas when it passes through the flameproof barrier/ arrestor. Furthermore, the coating of the flameproof bather allows the response time to be reduced from minutes (as can be found in commercially available sensors with sinters without the protective coating), to the order of seconds when the coating is present. A further advantage is that the invention improves the resistance of the detector to various environmental and chemical elements resulting in increased lifetime of many components of the sensor.

It is also found that the costs associated with the plasma coating of a sinter, and optionally other components of a gas detector, are less than those associated with the introduction of intrinsically safe electronics. Therefore, the present invention provides a cost effective mechanism for improving the flameproof bather within a gas detector.

1-10. (canceled)

11. A gas sensor having a flameproof barrier, the flameproof barrier comprising:
a porous element through which gas passes, wherein the porous element is coated with a protective layer, the protective layer configured to promote gas transport through the porous element.

12. The gas sensor of claim 11, wherein a thickness of the protective layer is less than 5 μm.

13. The gas sensor of claim 11, wherein the thickness of the protective layer is less than 1 μm.

14. The gas sensor of claim 11, wherein the thickness of the protective layer is equal to or less than one nanometer.

15. The gas sensor of claim 11, wherein the protective layer is applied to the porous element as a plasma coating.

16. The gas sensor of claim 11, wherein the protective layer is applied to the porous element using electrolytic deposition metal organic chemical deposition (MOCVD).

17. The gas sensor of claim 11, wherein the protective layer is applied to the porous element using chemical vapor deposition (CVD).

18. The gas sensor of claim 11, wherein the protective layer is a hydrophobic layer.

19. The gas sensor of claim 11, wherein the protective layer is selected from a group comprising SiO, 1H, 2H, 2H-heptadecafluorodecylacrylate, titanium dioxide, silicon monoxide, tin dioxide, and indium tin oxide.

20. The gas sensor of claim 19, wherein the protective layer is a chemical analogue of at least one selection from the group.

21. The gas sensor of claim 11, wherein the protective layer is selected wherein the protective layer promotes the transport of at least one of the following gases: H₂S, SO₂, NO, PH₃, O₃, HCl, HCN, and THF.

22. The gas sensor of claim 11, wherein the porous element is a porous sinter.

23. The gas sensor of claim 11, wherein the porous element is a flame arrestor.

24. The gas sensor of claim 11, wherein the protective layer is coated on both an inner surface and an outer surface of the porous element.

25. A gas detector comprising:
a gas sensor;
a flameproof barrier disposed on the gas sensor, the flameproof barrier including a porous element through which gas passes, wherein the porous element is coated with a hydrophobic protective layer configured to promote gas transport through the porous element.

26. The gas detector of claim 25, wherein the porous element is a porous sinter.

27. The gas detector of claim 25, wherein the pOTOUS element is a flame arrestor.

28. The gas detector of claim 25, wherein pipes, pumps, and supports of the gas detector are coated with the protective layer.
